(19) United States
(12) Patent Application Publication
(10) Pub. No.: US 2006/0142591 A1
(43) Pub. Date: Jun. 29, 2006

(54) DAPH analogs and inhibition of protein aggregation

(76) Inventors: Susan Lindquist, Chestnut Hill, MA (US); James Shorter, Cambridge, MA (US); Andrew Steele, Cambridge, MA (US); Martin Duennwald, Cambridge, MA (US); Vernon M. Ingram, Cambridge, MA (US); Stephen L. Buchwald, Newton, MA (US); Edward Hennessy, Roslindale, MA (US)

Correspondence Address: WOLF GREENFIELD & SACKS, PC;FEDERAL RESERVE PLAZA, 600 ATLANTIC AVENUE, BOSTON, MA, 02210-2206, US

(21) Appl. No.: 11/258,391

(22) Filed: Oct. 25, 2005

Related U.S. Application Data
(60) Provisional application No. 60/622,145, filed on Oct. 25, 2004.
(60) Provisional application No. 60/632,377, filed on Dec. 2, 2004.

(51) Int. Cl. C07D 209/48

(52) U.S. Cl. 548/476

(57) ABSTRACT

The invention relates to compounds which inhibit aggregation of proteins or peptides, or disaggregate protein aggregates, and thereby antagonize the toxic effects of such aggregates. The invention also relates to methods for using such compounds and methods for discovering compounds that inhibit protein or peptide aggregation.

### RELATED APPLICATIONS

This application claims the benefit under 35 U.S.C. §119(e) of U.S. provisional application Ser. No. 60/622,145, filed Oct. 25, 2004 and of U.S. provisional application Ser. No. 60/632,377, filed Dec. 2, 2004, the disclosures of which are incorporated by reference herein.

### FIELD OF THE INVENTION

The invention relates to compounds that are analogs of 4,5-dianilinophthalimide (DAPH), which are useful inter alia for inhibiting aggregation of proteins or peptides and thereby antagonizing the toxic effects of such aggregates. The invention also relates to methods for using such compounds.

### BACKGROUND OF THE INVENTION

A recent report by Blanchard et al. (Proc. Natl. Acad. Sci. USA 101:14326-14332, 2004) shows that certain compounds, including 4,5-dianilinophthalimide (DAPH), can disaggregate amyloid β (Aβ) fibers, which correlate with Alzheimer's disease.

DAPH is known as a tyrosine kinase inhibitor, particularly of the epidermal growth factor receptor, at least in vitro (Buchdunger et al., Proc. Natl. Acad. Sci. USA 91:2334-2338, 1994 (partial retraction at Proc. Natl. Acad. Sci. USA 95:12069, 1998); Trinks et al., J. Med. Chem. 37:1015-1027, 1994; U.S. Pat. Nos. 5,491,144 and 5,663,336).

It has long been known that certain proteins or peptides aggregate to form oligomers and higher order protein fiber structures. These fibers, also known as amyloid fibrils, can have deleterious effects when deposited outside or inside of cells. Protein aggregation is now recognized as important in a variety of neurodegenerative and other disorders, as well as central to a number of normal physiological processes, such as memory and biofilm formation.

For the treatment of protein aggregation disorders, it would be advantageous to obtain compounds having disaggregation properties (including inhibition of aggregation).

### SUMMARY OF THE INVENTION

It now has been determined that aggregates of peptides, such as amyloid β, and proteins such as amyloid fibrils of prion proteins, can be disaggregated using DAPH and analogs of DAPH. It also has been determined that DAPH and DAPH analogs can be used to inhibit aggregation of these proteins, such that less aggregation or even no aggregation takes place, i.e., less or no formation or protein aggregates or protein fibers. Furthermore, disaggregated monomeric proteins exhibit reduced aggregation under conditions that permit aggregation.

The invention involves in one aspect novel DAPH analogs and compositions containing these analogs. In another aspect, the invention involves methods and compositions for disaggregating protein oligomers and/or protein fibers. In another aspect, the invention involves methods and compositions for the treatment of conditions caused by protein oligomer and/or protein fiber aggregates by contacting protein oligomer and/or protein fiber aggregates with compounds which disaggregate the protein oligomers or the protein fibers. In a further aspect, the invention involves methods and compositions useful for inhibiting protein oligomer and/or protein fiber aggregation. The invention involves in another aspect methods for inhibiting biofilm formation by contacting the biofilm with compounds which disaggregate the protein oligomers or the protein fibers.

According to one aspect of the invention, novel DAPH analogs are provided. In one embodiment, the invention provides 5,6-Bis-(phenylamino)-2-(4-methoxy-phenyl)-isoindole-1,3-dione (DAPH-2) having the structure: 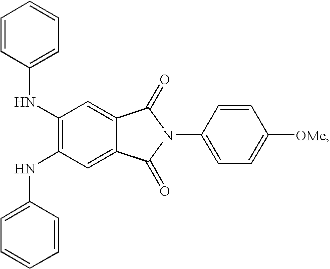
a salt thereof or a solvate thereof.

In another embodiment, the invention provides 5,6-Bis-(2-chloro-phenylamino)-2-(4-methoxyphenyl)-isoindole-1,3-dione (DAPH-3) having the structure: 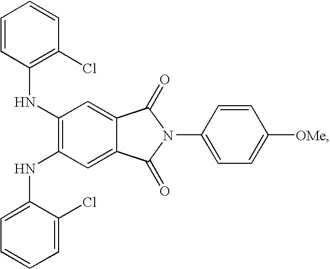
a salt thereof or a solvate thereof.

In another embodiment, the invention provides 5,6-Bis-(3,5-dimethyl-phenylamino)-2-(4-methoxyphenyl)-isoindole-1,3-dione (DAPH-4) having the structure: 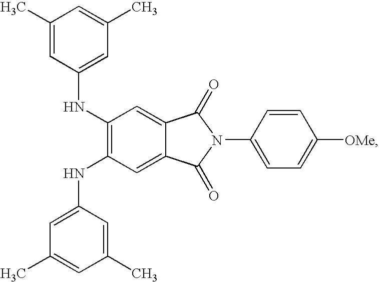
a salt thereof or a solvate thereof.

In another embodiment, the invention provides 5,6-Bis-(4-fluoro-phenylamino)-isoindole-1,3-dione (DAPH-7) having the structure: 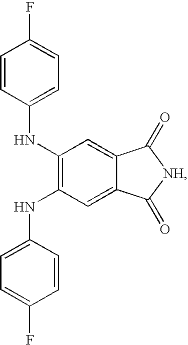
a salt thereof or a solvate thereof.

In another embodiment, the invention provides 5,6-Bis-(4-nitro-phenylamino)-isoindole-1,3-dione (DAPH-10) having the structure: 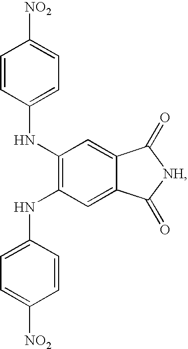
a salt thereof or a solvate thereof.

In another embodiment, the invention provides 4,5-Bis-phenylamino-phthalic acid dimethyl ester (DAPH-11) having the structure: 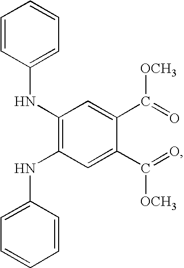
a salt thereof or a solvate thereof.

In another embodiment, the invention provides 5,6-Bis-(4-methoxy-phenylamino)-isoindole-1,3-dione (DAPH-12) having the structure: 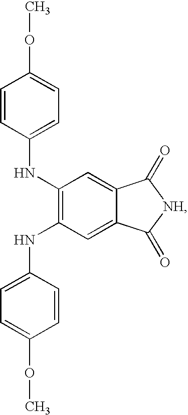
a salt thereof or a solvate thereof.

In still another embodiment, the invention provides pharmaceutical compositions comprising any one or more of the foregoing DAPH analogs, salts thereof or solvates thereof and a pharmaceutically acceptable carrier. Kits comprising one or more containers containing one or more DAPH analogs, salts thereof, solvates thereof and/or pharmaceutical compositions also are provided.

According to a further aspect of the invention, methods for disaggregating protein oligomers and/or protein fibers are provided. The methods include contacting protein oligomers and/or protein fibers with one or more compounds selected from the group consisting of 4,5-dianilinophthalimide (DAPH), DAPH analogs, salts thereof and solvates thereof, in an amount effective to disaggregate the protein oligomers or the protein fibers is provided. In one embodiment the disaggregation reduces the toxicity of the protein oligomers and/or protein fibers. In a second embodiment the compound is 4,5-dianilinophthalimide (DAPH), a salt thereof or a solvate thereof. In another embodiment the compound is a DAPH analog (preferably 5,6-Bis-(4-fluoro-phenylamino)-isoindole-1,3-dione (DAPH-7) or 5,6-Bis-(4-methoxy-phenylamino)-isoindole-1,3-dione (DAPH-12) or bisindolylmaleimide IV (DAPH-13)), a salt thereof or a solvate thereof.

In a further embodiment the protein is selected from the group consisting of amyloid β (Aβ), huntingtin, PrP prion, α-synuclein, cytoplasmic polyadenylation element binding protein (CPEB), yeast Sup35 NM protein (PSI⁺ prion), synphilin, transthyretin, tau, ataxin 1, ataxin 3, atrophin, cystatin B, cystic fibrosis transmembrane conductance regulator (CFTR), the variable domain of immunoglobulin light chains, insulin, leptin, α-lactalbumin, phosphodiesterase γ, prothymosin α and apo-lipoprotein E4, and androgen receptor. In another embodiment the protein fibers are amyloid fibrils.

In another aspect of the invention, methods for inhibiting aggregation of protein oligomers and/or protein fibers are provided. The methods include contacting protein oligomers and/or protein fibers with one or more compounds selected from the group consisting of 4,5-dianilinophthalimide (DAPH), DAPH analogs, salts thereof and solvates thereof, in an amount effective to disaggregate the protein oligomers or the protein fibers is provided. In one embodiment the inhibition of aggregation reduces toxicity of the protein oligomers and/or protein fibers. In a second embodiment the compound is 4,5-dianilinophthalimide (DAPH), a salt thereof or a solvate thereof. In another embodiment the compound is bisindolylmaleimide IV, a salt thereof or a solvate thereof. In another embodiment the compound is a DAPH analog (preferably 5,6-Bis-(4-fluoro-phenylamino)-isoindole-1,3-dione (DAPH-7) or 5,6-Bis-(4-methoxy-phenylamino)-isoindole-1,3-dione (DAPH-12) or bisindolylmaleimide IV (DAPH-13)), a salt thereof or a solvate thereof.

In a further embodiment the protein is selected from the group consisting of amyloid β (Aβ), huntingtin, PrP prion, α-synuclein, cytoplasmic polyadenylation element binding protein (CPEB), yeast Sup35 NM protein (PSI⁺ prion), synphilin, transthyretin, tau, ataxin 1, ataxin 3, atrophin, cystatin B, cystic fibrosis transmembrane conductance regulator (CFTR), the variable domain of immunoglobulin light chains, insulin, leptin, α-lactalbumin, phosphodiesterase γ, prothymosin α and apo-lipoprotein E4, and androgen receptor. In yet another embodiment the protein fibers are amyloid fibrils.

According to a further aspect of the invention, additional methods for disaggregating protein oligomers and/or protein fibers are provided. The methods include contacting protein oligomers and/or protein fibers with (a) 4,5-dianilinophthalimide (DAPH), salts thereof or solvates thereof and (b) one or more DAPH analogs, salts thereof or solvates thereof, in a combined amount effective to disaggregate the protein oligomers or the protein fibers. Preferred DAPH analogs include 5,6-Bis-(4-fluoro-phenylamino)-isoindole-1,3-dione (DAPH-7), 5,6-Bis-(4-methoxy-phenylamino)-isoindole-1,3-dione (DAPH-12) and bisindolylmaleimide IV (DAPH-13). In one embodiment the disaggregation reduces toxicity of the protein oligomers and/or protein fibers. In a second embodiment the protein is selected from the group consisting of amyloid β (Aβ), huntingtin, PrP prion, α-synuclein, cytoplasmic polyadenylation element binding protein (CPEB), yeast Sup35 NM protein (PSI⁺ prion), synphilin, transthyretin, tau, ataxin 1, ataxin 3, atrophin, cystatin B, cystic fibrosis transmembrane conductance regulator (CFTR), the variable domain of immunoglobulin light chains, insulin, leptin, α-lactalbumin, phosphodiesterase γ, prothymosin α and apo-lipoprotein E4, and androgen receptor. In another embodiment the protein fibers are amyloid fibrils.

In another aspect of the invention, methods for inhibiting aggregation of protein oligomers and/or protein fibers are provided. The methods include contacting protein oligomers and/or protein fibers with (a) 4,5-dianilinophthalimide (DAPH), salts thereof or solvates thereof and (b) one or more DAPH analogs, salts thereof or solvates thereof, in a combined amount effective to disaggregate the protein oligomers or the protein fibers. Preferred DAPH analogs include 5,6-Bis-(4-fluoro-phenylamino)-isoindole-1,3-dione (DAPH-7), 5,6-Bis-(4-methoxy-phenylamino)-isoindole-1,3-dione (DAPH-12) and bisindolylmaleimide IV (DAPH-13). In one embodiment the inhibition of aggregation reduces toxicity of the protein oligomers and/or protein fibers. In a second embodiment the protein is selected from the group consisting of amyloid β (Aβ), huntingtin, PrP prion, α-synuclein, cytoplasmic polyadenylation element binding protein (CPEB), yeast Sup35 NM protein (PSI⁺ prion), synphilin, transthyretin, tau, ataxin 1, ataxin 3, atrophin, cystatin B, cystic fibrosis transmembrane conductance regulator (CFTR), the variable domain of immunoglobulin light chains, insulin, leptin, α-lactalbumin, phosphodiesterase γ, prothymosin α and apo-lipoprotein E4, and androgen receptor. In another embodiment the protein fibers are amyloid fibrils.

According to a further aspect of the invention, methods for treating a subject having a protein aggregation disorder are provided. The methods include administering to a subject in need of such treatment one or more compounds selected from the group consisting of 4,5-dianilinophthalimide (DAPH), DAPH analogs, salts thereof and solvates thereof, in an amount effective to disaggregate protein oligomers or protein fibers. Preferably the disaggregation reduces toxicity of the protein oligomers and/or protein fibers.

In one embodiment the compound is 4,5-dianilinophthalimide (DAPH), a salt thereof or a solvate thereof. In another embodiment the compound is a DAPH analog (preferably 5,6-Bis-(4-fluoro-phenylamino)-isoindole-1,3-dione (DAPH-7) or 5,6-Bis-(4-methoxy-phenylamino)-isoindole-1,3-dione (DAPH-12) or bisindolylmaleimide IV (DAPH-13)), a salt thereof or a solvate thereof.

In another embodiment the protein is selected from the group consisting of amyloid β (Aβ), huntingtin, PrP prion, α-synuclein, cytoplasmic polyadenylation element binding protein (CPEB), yeast Sup35 NM protein (PSI⁺ prion), synphilin, transthyretin, tau, ataxin 1, ataxin 3, atrophin, cystatin B, cystic fibrosis transmembrane conductance regulator (CFTR), the variable domain of immunoglobulin light chains, insulin, leptin, α-lactalbumin, phosphodiesterase γ, prothymosin α and apo-lipoprotein E4, and androgen receptor. In a further embodiment the protein fibers are amyloid fibrils.

In yet another embodiment the protein aggregation disorder is selected from the group consisting of Alzheimer's disease, α-synucleinopathies; Parkinson's disease; Huntington's Disease; prion related diseases; tauopathies; Type II Diabetes Mellitus associated with islet amyloid polypeptide (IAPP); and neurodegenerative diseases associated with intracellular and/or intraneuronal aggregates of proteins with polyglutamine, polyalanine or other repeats arising from pathological expansions of tri- or tetra-nucleotide elements within corresponding genes. In still another embodiment the subject is free of symptoms otherwise calling for treatment with the composition.

In a further aspect of the invention, methods for treating a subject having a protein aggregation disorder are provided. The methods include administering to a subject in need of such treatment one or more compounds selected from the group consisting of 4,5-dianilinophthalimide (DAPH), DAPH analogs, salts thereof and solvates thereof, in an amount effective to inhibit aggregation of protein oligomers and/or protein fibers. Preferably the inhibition of aggregation reduces toxicity of the protein oligomers and/or protein fibers.

In one embodiment the compound is 4,5-dianilinophthalimide (DAPH), a salt thereof or a solvate thereof. In another embodiment the compound is a DAPH analog (preferably 5,6-Bis-(4-fluoro-phenylamino)-isoindole-1,3-dione (DAPH-7) or 5,6-Bis-(4-methoxy-phenylamino)-isoindole-1,3-dione (DAPH-12) or bisindolylmaleimide IV (DAPH-13)), a salt thereof or a solvate thereof.

In another embodiment the protein is selected from the group consisting of amyloid β (Aβ), huntingtin, PrP prion, α-synuclein, cytoplasmic polyadenylation element binding protein (CPEB), yeast Sup35 NM protein (PSI⁺ prion), synphilin, transthyretin, tau, ataxin 1, ataxin 3, atrophin, cystatin B, cystic fibrosis transmembrane conductance regulator (CFTR), the variable domain of immunoglobulin light chains, insulin, leptin, α-lactalbumin, phosphodiesterase γ, prothymosin α and apo-lipoprotein E4, and androgen receptor. In a further embodiment the protein fibers are amyloid fibrils.

In yet another embodiment the protein aggregation disorder is selected from the group consisting of Alzheimer's disease, α-synucleinopathies; Parkinson's disease; Huntington's Disease; prion related diseases; tauopathies; Type II Diabetes Mellitus associated with islet amyloid polypeptide (IAPP); and neurodegenerative diseases associated with intracellular and/or intraneuronal aggregates of proteins with polyglutamine, polyalanine or other repeats arising from pathological expansions of tri- or tetra-nucleotide elements within corresponding genes. In still another embodiment the subject is free of symptoms otherwise calling for treatment with the composition.

In another aspect of the invention, methods for treating a subject having a protein aggregation disorder are provided. The methods include administering to a subject in need of such treatment (a) 4,5-dianilinophthalimide (DAPH), salts thereof or solvates thereof and (b) one or more a DAPH analogs, salts thereof or solvates thereof, in a combined amount effective to disaggregate protein oligomers or protein fibers. In one embodiment the disaggregation reduces toxicity of the protein oligomers and/or protein fibers. Preferred DAPH analogs include 5,6-Bis-(4-fluoro-phenylamino)-isoindole-1,3-dione (DAPH-7), 5,6-Bis-(4-methoxy-phenylamino)-isoindole-1,3-dione (DAPH-12) and bisindolylmaleimide IV (DAPH-13).

In a second embodiment the protein is selected from the group consisting of amyloid β (Aβ), huntingtin, PrP prion, α-synuclein, cytoplasmic polyadenylation element binding protein (CPEB), yeast Sup35 NM protein (PSI⁺ prion), synphilin, transthyretin, tau, ataxin 1, ataxin 3, atrophin, cystatin B, cystic fibrosis transmembrane conductance regulator (CFTR), the variable domain of immunoglobulin light chains, insulin, leptin, α-lactalbumin, phosphodiesterase γ, prothymosin α and apo-lipoprotein E4, and androgen receptor. In another embodiment the protein fibers are amyloid fibrils. In a further embodiment the protein aggregation disorder is selected from the group consisting of Alzheimer's disease; tauopathy; α-synucleinopathy; Parkinson's disease; Huntington's Disease; prion related diseases; Type II Diabetes Mellitus associated with islet amyloid polypeptide (IAPP); and neurodegenerative diseases associated with intracellular and/or intraneuronal aggregates of proteins with polyglutamine, polyalanine or other repeats arising from pathological expansions of tri- or tetra-nucleotide elements within corresponding genes. In yet another embodiment the subject is free of symptoms otherwise calling for treatment with the composition.

In still another aspect of the invention, methods for treating a subject having a protein aggregation disorder are provided. The methods include administering to a subject in need of such treatment (a) 4,5-dianilinophthalimide (DAPH), salts thereof or solvates thereof and (b) one or more DAPH analogs, salts thereof or solvates thereof, in a combined amount effective to inhibit aggregation of protein oligomers and/or protein fibers. In one embodiment the inhibition of aggregation reduces toxicity of the protein oligomers and/or protein fibers. Preferred DAPH analogs include 5,6-Bis-(4-fluoro-phenylamino)-isoindole-1,3-dione (DAPH-7), 5,6-Bis-(4-methoxy-phenylamino)-isoindole-1,3-dione (DAPH-12) and bisindolylmaleimide IV (DAPH-13).

In a second embodiment the protein is selected from the group consisting of amyloid β (Aβ), huntingtin, PrP prion, α-synuclein, cytoplasmic polyadenylation element binding protein (CPEB), yeast Sup35 NM protein (PSI⁺ prion), synphilin, transthyretin, tau, ataxin 1, ataxin 3, atrophin, cystatin B, cystic fibrosis transmembrane conductance regulator (CFTR), the variable domain of immunoglobulin light chains, insulin, leptin, α-lactalbumin, phosphodiesterase γ, prothymosin α and apo-lipoprotein E4, and androgen receptor. In another embodiment the protein fibers are amyloid fibrils.

In a further embodiment the protein aggregation disorder is selected from the group consisting of Alzheimer's disease, α-synucleinopathies; Parkinson's disease; Huntington's Disease; prion related diseases; tauopathies; Type II Diabetes Mellitus associated with islet amyloid polypeptide (IAPP); and neurodegenerative diseases associated with intracellular and/or intraneuronal aggregates of proteins with polyglutamine, polyalanine or other repeats arising from pathological expansions of tri- or tetra-nucleotide elements within corresponding genes. In yet another embodiment the subject is free of symptoms otherwise calling for treatment with the composition.

In another aspect of the invention, compositions that include a combination of 4,5-dianilinophthalimide (DAPH), salts thereof or solvates thereof, and one or more DAPH analogs, salts thereof or solvates thereof is provided. In one embodiment the composition further comprises a pharmaceutically acceptable carrier. In a second embodiment the compounds are present in a combined amount effective to inhibit aggregation of protein oligomers and/or protein fibers or disaggregate protein oligomers and/or protein fibers. Preferred DAPH analogs include 5,6-Bis-(4-fluoro-phenylamino)-isoindole-1,3-dione (DAPH-7), 5,6-Bis-(4-methoxy-phenylamino)-isoindole-1,3-dione (DAPH-12) and bisindolylmaleimide IV (DAPH-13).

In another aspect of the invention, methods for inhibiting biofilm formation are provided. The methods include contacting an organism that forms a biofilm with one or more compounds selected from the group consisting of 4,5-dianilinophthalimide (DAPH), DAPH analogs, salts thereof and solvates thereof, in an amount effective to inhibit aggregation of protein oligomers or protein fibers in the biofilm. Preferred DAPH analogs include 5,6-Bis-(4-fluoro-phenylamino)-isoindole-1,3-dione (DAPH-7) and 5,6-Bis-(4-methoxy-phenylamino)-isoindole-1,3-dione (DAPH-12) and bisindolylmaleimide IV (DAPH-13).

In yet another aspect of the invention, methods for disaggregating biofilms are provided. The methods include contacting the biofilm with one or more compounds selected from the group consisting of 4,5-dianilinophthalimide (DAPH), analogs of DAPH, salts thereof and solvates thereof, in an amount effective to disaggregate the biofilm. Preferred DAPH analogs include 5,6-Bis-(4-fluoro-phenylamino)-isoindole-1,3-dione (DAPH-7) and 5,6-Bis-(4-methoxy-phenylamino)-isoindole-1,3-dione (DAPH-12) and bisindolylmaleimide IV (DAPH-13).

The use of the foregoing compositions in the preparation of medicaments also is provided. These and other objects and features of the invention are described in greater detail below.

### BRIEF DESCRIPTION OF THE DRAWINGS

FIG. 1 shows that DAPH antagonizes NM fiber assembly.

FIG. 2 shows that DAPH and DAPH analogs partially dissolve NM fibers as measured by Congo Red binding.

FIG. 3 shows that DAPH and DAPH analogs partially dissolve NM fibers as measured by ThT fluorescence.

FIG. 4 shows Kaplan-Meier survival plots of mice infected with brain homogenates of mice infected with the RML strain of mouse scrapie.

### DETAILED DESCRIPTION OF THE INVENTION

The invention provides a series of novel analogs of DAPH having surprising activity to disaggregate proteins and peptides, and to inhibit the aggregation of proteins and peptides. These compounds, along with related pharmaceutical compositions and methods, are useful in the treatment and prevention of protein aggregations disorders and diseases.

We have synthesized a series of analogs of DAPH (4,5-dianilinophthalimide) in order to identify compounds that retain the ability to disaggregate or inhibit aggregation of proteins and peptides. The DAPH analogs are described in Table 1 by chemical name and molecular weight. Included in the set of analogs described in Table 1 are several commercially available DAPH analog molecules; these are designated DAPH-5, DAPH-6, DAPH-8 and DAPH-9.

TABLE 1
DAPH and DAPH Analogs (DAPH-2-DAPH-13)

| Name | Compound | MW |
|---|---|---|
| DAPH | 4,5-dianilinophthalimide (Sigma D210) = | 329.4 |
| | 5,6-Bis-phenylamino-isoindole-1,3-dione | |
| DAPH-2 | 5,6-Bis-(phenylamino)2-(4-methoxy-phenyl)- | 435.48 |
| | isoindole-1,3-dione (Anisole-DAPH) | |
| DAPH-3 | o-Chloro compound: 5,6-Bis-(2-chloro- | 491.58 |
| | phenylamino)-2-(4-methoxyphenyl)-isoindole-1,3- | |
| | dione (anisole derivative) | |
| DAPH-4 | dimethyl compound: 5,6-Bis-(3,5-dimethyl- | 504.36 |
| | phenylamino)-2-(4-methoxyphenyl)-isoindole-1,3- | |
| | dione (anisole derivative) | |
| DAPH-5 | 3-amino-phthalimide (Sigma 524794) | 162.15 |
| DAPH-6 | N-Amino-phthalimide (Sigma 178314) | 162.15 |
| DAPH-7 | 5,6-Bis-(4-fluoro-phenylamino)-isoindolo-1,3-dione | 365.33 |
| DAPH-8 | Phthalimide (Sigma 240230) | 147.13 |
| DAPH-9 | 4,5-Dichlorophthalimide (Sigma 422665) | 216.02 |
| DAPH-10 | 5,6-Bis-(4-nitro-phenylamino)-isoindolo-1,3-dione | 419.35 |
| | (nitro derivative) | |
| DAPH-11 | diester compound: 4,5-Bis-phenylamino-phthalic | 376.41 |
| | acid dimethyl ester | |
| DAPH-12 | dimethoxy DAPH: 5,6-Bis-(4-methoxy- | 389.4 |
| | phenylamino)-isoindole-1,3-dione | |
| DAPH-13 | Bisindolylmaleimide IV; 5,6-Bis-(4-dimethylamino- | 327.34 |
| | phenylamino)-isoindole-1,3-dione; arcyriarubin A; | |
| | 2,3-bis(1H-indol-3-yl)maleimide) | |

Typically the molecules are stored at −20° C. in DMSO at a convenient concentration, e.g., 10 mM. The structures of DAPH and the DAPH analogs are shown below:

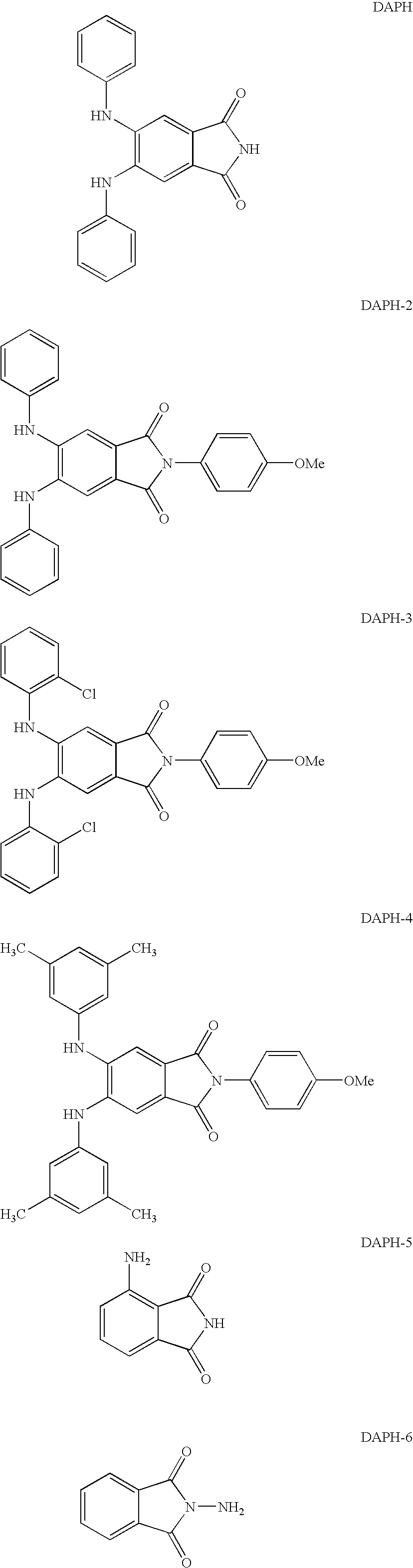

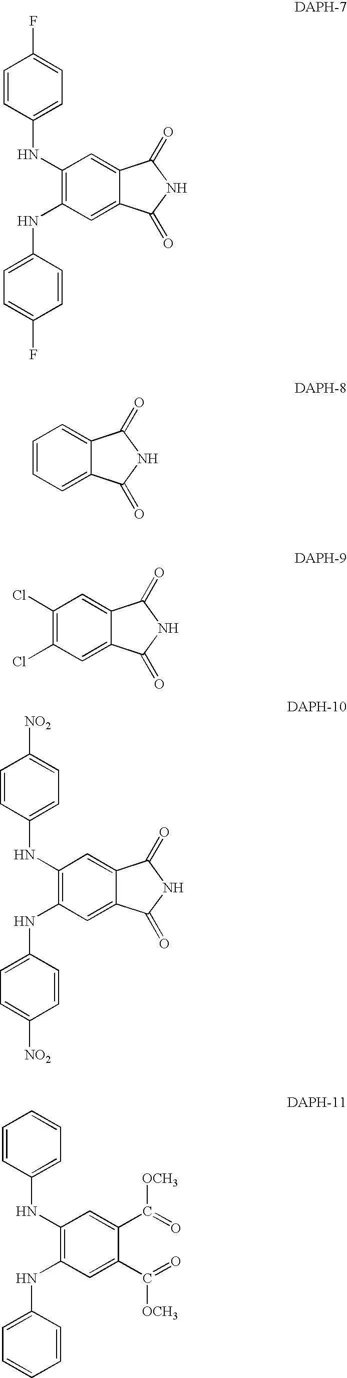

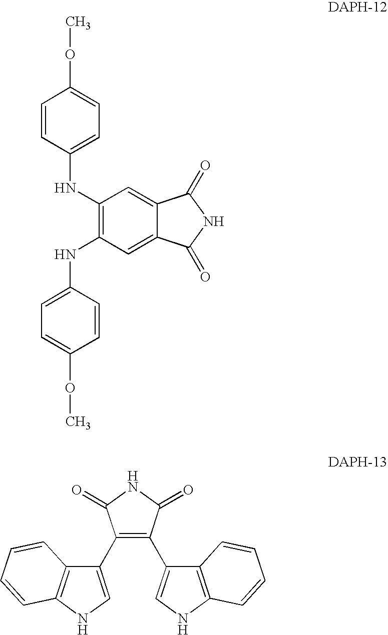

Where the DAPH analogs according to this invention have at least one chiral center, they may accordingly exist as enantiomers. Where the DAPH analogs possess two or more chiral centers, they may additionally exist as diastereomers. It is to be understood that all such isomers and mixtures thereof are encompassed within the scope of the present invention. Furthermore, some of the crystalline forms for DAPH and the DAPH analogs may exist as polymorphs and as such are intended to be included in the present invention. In addition, some of the compounds may form solvates with water (i.e., hydrates) or common organic solvents, and such solvates are also intended to be encompassed within the scope of this invention.

DAPH and the DAPH analogs of the invention can be isolated and used as free bases. They can also be isolated and used as pharmaceutically acceptable salts or solvates. Examples of such salts include hydrobromic, hydroiodic, hydrochloric, perchloric, sulfuric, maleic, fumaric, malic, tartaric, citric, benzoic, mandelic, methanesulfonic, hydroethanesulfonic, benzenesulfonic, oxalic, palmoic, 2-naphthalenesulfonic, p-toluenesulfonic, cyclohexanesulfamic and saccharic.

The invention further provides methods of disaggregating or inhibiting the aggregation of protein oligomers and/or protein fibers including contacting protein oligomers and/or protein fibers with an effective amount of one or more of DAPH and DAPH analogs described herein. The contacting can be performed, for example, in vitro, ex vivo, or in vivo. Similarly, methods for inhibiting aggregation of protein oligomers and/or protein fibers are provided.

We have determined that DAPH and DAPH analog molecules inhibit amyloid fiber formation by amyloid β peptides (Aβ) and also effectively inhibit aggregation (also known as fibrillization) by a variety of other proteins involved in diseases and disorders of protein aggregation, including prion proteins, α-synuclein and polyglutamine proteins such as huntingtin. In addition, these molecules can also disaggregate existing protein fibers and protein oligomers to a monomeric form. The disaggregated proteins show a reduced ability to seed further aggregates/amyloid fibrils.

Aggregate formation generally involves proteins with a relatively high content of β-sheet forming sequence. Perhaps the best-known example of protein aggregation is the aggregation of amyloid β (Aβ). β-sheet forming sequences present in the amyloid precursor protein (APP) can be released by proteolysis, and the peptides with β-sheet forming sequences (e.g., Aβ42) thus released can aggregate. Aggregated Aβ forms the amyloid plaques that are characteristic of Alzheimer's disease.

Protein aggregation is not limited to peptides, however. Under certain conditions, a variety of proteins can form multimers or aggregates inside and outside of cells, often in the form of oligomers or fibrils frequently referred to generically as “amyloid fibrils”. A common cause of aggregation is misfolding of the protein. Misfolded proteins appear to be more likely to aggregate into oligomers and higher order multimeric structures. See, for example, Ellis and Pinheiro, Nature 416:483-484, 2002 for a brief overview of the contribution of protein misfolding to protein aggregation.

The problem of protein aggregation to form amyloid fibrils has been recognized in a diverse set of diseases, disorders and conditions. Prion proteins are now recognized to form amyloid fibrils, which have implications for neurodegenerative diseases such as Creutzfeldt-Jakob disease, as well as long-term memory (via aggregation of the CPEB protein (Si et al., Cell. 115(7):879-891, 2003)). A number of neurodegenerative diseases and other degenerative disease are associated with the appearance of amyloid fibrils, including Alzheimer's disease, Parkinson's disease, Huntington's disease, Familial amyloid polyneuropathies, Familial dementias and others (see, e.g., Huff et al., Current Opin. Struct. Biol. 13 :674-682, 2003). The foregoing are referred to herein as “protein aggregation disorders” and the like. In addition, amyloid fibrils are recognized as contributing to biofilm formation (Huff et al., 2003).

It is believed that proteins are toxic when aggregated at least in part because they bind together to form oligomers or amyloid fibrils linked together by binding of β-sheet structures of the proteins. The aggregated proteins can have a variety of toxic effects, thereby causing or contributing to disease. Thus, compounds which prevent binding of protein oligomers or protein fiber aggregates, or which reduce the formation or size of the aggregates, such as fibrils can be useful for reducing the toxicity of protein aggregates.

According to aspects of the invention, methods and compositions for disaggregating such protein oligomers and/or protein fibers are presented. In a further aspect, the invention provides methods and compositions for inhibiting protein oligomer and/or protein fiber aggregate formation. In another aspect, the invention provides methods and compositions for treating a subject having a protein disaggregation disorder. In particular, the toxicity of protein aggregates can be ameliorated using the compounds of the invention.

The invention thus involves in one aspect methods for disaggregating protein oligomers and/or protein fibers using DAPH and/or DAPH analog(s) in an amount effective to disaggregate the protein oligomers or the protein fibers. The disaggregation of the protein oligomers and/or protein fibers reduces the toxicity of the protein oligomers and/or protein fibers. In one aspect the proteins include but are not limited to amyloid β peptides (Aβ), huntingtin and other polyglutamine proteins, PrP prion, α-synuclein, cytoplasmic polyadenylation element binding protein (CPEB), yeast Sup35 NM protein (PSI⁺ prion), synphilin, transthyretin, tau, ataxin 1, ataxin 3, atrophin, cystatin B, cystic fibrosis transmembrane conductance regulator (CFTR), the variable domain of immunoglobulin light chains, insulin, leptin, α-lactalbumin, phosphodiesterase γ, prothymosin α and apo-lipoprotein E4, and androgen receptor. In a preferred embodiment the protein fibers are amyloid fibrils.

Protein oligomer and protein fiber aggregate formation can be determined directly, e.g., by observation of the extent of protein oligomer and protein fiber aggregate formation by microscopy (e.g., electron microscopy) or by dye binding (e.g., Congo Red binding or Thioflavin T binding), or indirectly, e.g., by determination of the effects of protein oligomer and protein fiber aggregate formation, such as a change in cytotoxicity. Other methods for determining the extent or effects of protein oligomer and protein fiber aggregate formation will be apparent to one of ordinary skill in the art. Assays such as Congo Red binding and Thioflavin T binding are particularly useful.

The invention includes the use of one or more of DAPH and DAPH analogs for disaggregating protein oligomer and/or protein fiber aggregates. DAPH analogs useful in the methods include but are not limited to DAPH-2, DAPH-3, DAPH-4, DAPH-5, DAPH-6, DAPH-7, DAPH-8, DAPH-9, DAPH-10, DAPH-11, DAPH-12 and DAPH-13, salts thereof and solvates thereof.

It is also contemplated that any DAPH analog of the invention can be used in combination with another one or more DAPH analogs (previously known or novel analogs of the invention), with DAPH, or with other molecules that are efficacious in disaggregating or inhibiting the aggregation of proteins and/or peptides. The combination of compounds is intended to include salts of the compounds and solvates of the foregoing molecules. The use of combinations of DAPH and/or DAPH analogs, particularly the novel DAPH analogs, with other molecules permits, for example, the use of reduced amounts of each molecule, such that the side effects of each molecule can be minimized. The use of combinations of DAPH and/or DAPH analogs, particularly the novel DAPH analogs, with other molecules also permits synergistic inhibition of aggregation and/or disaggregation of proteins and/or peptides.

Changes to the structure of a DAPH analog molecule (of DAPH) to form variants or analogs of such a molecule can be made according to established principles in the art. Such changes can be made to increase the therapeutic efficacy of the DAPH analog, reduce side effects of the DAPH analog, increase or decrease the hydrophobicity or hydrophilicity, and the like. For example, Traxler et al. (J. Med. Chem. 38:2441-2448, 1995) demonstrated the effect that substitutions in different parts of DAPH have on its ability to inhibit tyrosine kinase and other protein kinases. Eliminating from the DAPH molecule its kinase-inhibitor capacity is expected to reduce toxicity complications. Changes to the structure include the addition of additional functional groups, such as for targeting the DAPH analog to a particular organ of a subject, and substitution of one or more portions of the DAPH analog.

The compounds (i.e., DAPH and/or DAPH analogs) used in the methods for disaggregating or inhibiting the protein oligomer and/or protein fiber aggregates may exist in different isomeric forms. The compounds may be used in the methods of the invention as a substantially isomerically-pure molecule, or as a mixture of isomers. Preferably, isomerically-pure compounds are used. Isomerically-pure, as used herein, means that one isomer will be present in an amount ranging from 51 to 100%, preferably, more than 80%, more preferably, more than 90%, even more preferably, more than 95%, and even more preferably, more than 99% pure with respect to the other isomer or isomers present, but not with respect to other impurities or compounds that may be present. Isomer, as used herein, may refer to an E or Z isomer, and R or S isomer, an enantiomer, a diastereomer, or, in the case of compounds with several diastereomers, a group of diastereomers, with respect to another group of diastereomers, which differ for example, with respect to just one stereocenter of the molecule.

Compounds (i.e., DAPH and/or DAPH analogs) which disaggregate and/or inhibit the ability of protein oligomers and/or protein fibers to form aggregates can be administered to a subject to treat a condition characterized by unwanted protein oligomer and/or protein fiber aggregates. Compounds are administered in an amount effective to disaggregate and/or inhibit formation of unwanted aggregates. By effective amount is meant an amount of a compound(s) that inhibits formation of new unwanted protein oligomers and/or protein fiber aggregates, modifies the structure of new or existing unwanted aggregates, or destabilizes existing unwanted aggregates such that the aggregates (e.g., amyloid fibrils) are disaggregated.

Conditions characterized by unwanted protein oligomer and/or protein fiber aggregate formation include Alzheimer's disease and other disorders that include aggregation of Aβ, α-synucleinopathies; Parkinson's disease; Huntington's disease; prion related diseases; tauopathies; Type II Diabetes Mellitus associated with islet amyloid polypeptide (IAPP); and neurodegenerative diseases associated with intracellular and/or intraneuronal aggregates of proteins with polyglutamine, polyalanine or other repeats arising from pathological expansions of tri- or tetra-nucleotide elements within corresponding genes.

Additional protein aggregation diseases include Amyotrophic Lateral Sclerosis; motor neuron disease; Spastic paraplegia; spinocerebellar ataxia, Freidrich's Ataxia; cerebrovascular diseases; Down's syndrome; head trauma with post-traumatic accumulation of amyloid beta peptide; Familial British Dementia; Familial Danish Dementia; Presenile Dementia with Spastic Ataxia; Cerebral Amyloid Angiopathy, British Type; Presenile Dementia With Spastic Ataxia Cerebral Amyloid Angiopathy, Danish Type; Familial encephalopathy with neuroserpin inclusion bodies (FENIB); Amyloid Polyneuropathy; Transthyretin amyloidosis; Inclusion Body myositis due to amyloid beta (Aβ) peptide; Familial and Finnish Type Amyloidosis; Systemic amyloidosis associated with multiple myeloma; Familial Mediterranean Fever; chronic infections and inflammations; and cystic fibrosis.

Tauopathies include argyrophilic grain dementia, corticobasal degeneration, dementia pugilistica, diffuse neurofibrillary tangles with calcification, frontotemporal dementia with parkinsonism, Hallervorden-Spatz disease, myotonic dystrophy, Niemann-Pick disease type C, non-Guamanian Motor Neuron disease with neurofibrillary tangles, Pick's disease, postencephalitic parkinsonism, prion protein cerebral amyloid angiopathy, progressive subcortical gliosis, progressive supranuclear palsy, subacute sclerosing panencephalitis, and tangle only dementia.

α-synucleinopathies include dementia with Lewy bodies, multiple system atrophy with glial cytoplasmic inclusions, Shy-Drager syndrome, striatonigral degeneration, olivopontocerebellar atrophy, neurodegeneration with brain iron accumulation type I, and olfactory dysfunction.

Prion related diseases include Creutzfeldt-Jakob disease, Gerstmann-Straussler-Scheinker disease, and variant Creutzfeldt-Jakob disease.

In addition, based on the observed disaggregation of NM protein, prion diseases can be treated. For example, it is believed to be possible to cure cells of yeast prions, and it is anticipated that similar results are possible in mammalian prion diseases. Further based on the effects demonstrated with the prion-like CPEB protein and its effects in long-term memory (Si et al., Cell. 115(7):879-891, 2003), it is contemplated to use DAPH and/or DAPH analogs and compositions described herein for memory treatments.

Biofilms are generally composed of bacterial communities attached to a surface and their inherent resistance to antimicrobial agents are a cause of many persistent and chronic bacterial infections. Biofilms of E. coli, for example, include curli, which are extracellular amyloid fibers that permit binding and colonization of many surfaces (Prigent-Combaret et al., Environ Microbiol. 2(4):450-464, 2000; Huff et al., 2003). Methods for inhibiting biofilm formation and for disaggregating biofilms are hence provided. The methods contemplate contacting an organism that forms a biofilm with one or more compounds (i.e., DAPH and DAPH analogs) in an amount effective to disaggregate the protein oligomers or the protein fibers. Methods for disaggregating biofilm formation by contacting the biofilm with one or more compounds in an amount effective to disaggregate the biofilm are also provided.

Effective amount is further contemplated to mean that protein oligomer and/or protein fiber aggregation is inhibited or reduced by at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

As used herein, a subject includes mammals, fish, birds and reptiles. A preferred subject is a mammal, preferably a veterinary animal. More preferably, the subject is a human. A subject is intended to include a subject having a protein aggregation disorder, a subject being diagnosed as having a protein aggregation disorder, a subject having symptoms of a protein aggregation disorder, and a subject at risk of developing a protein aggregation disorder.

DAPH and/or DAPH analogs which disaggregate or inhibit the formation of protein oligomer and/or protein fiber aggregates may be administered as part of a pharmaceutical composition. Such a pharmaceutical composition may include DAPH and/or the DAPH analogs in combination with any physiologically and/or pharmaceutically acceptable carriers which are known in the art. The compositions should be sterile and contain a therapeutically effective amount of DAPH and/or the DAPH analog(s) in a unit of weight or volume suitable for administration to a patient. The term “pharmaceutically acceptable” means a non-toxic material that does not interfere with the effectiveness of the biological activity of the active ingredients. The term “physiologically acceptable” refers to a nontoxic material that is compatible with a biological system such as a cell, cell culture, tissue, or organism. The characteristics of the carrier will depend on the route of administration. Physiologically and pharmaceutically acceptable carriers include diluents, fillers, salts, buffers, stabilizers, solubilizers, and other materials which are well known in the art.

When used therapeutically, DAPH and/or the DAPH analogs of the invention are administered in therapeutically effective amounts. In general, a therapeutically effective amount means that amount necessary to delay the onset of, inhibit the progression of, or halt altogether the particular condition being treated. Therapeutically effective amounts specifically will be those which desirably influence the existence or formation of aggregates of protein oligomer and/or protein fiber aggregates, and/or desirably influence the cytotoxic (or other) effects of such aggregates.

Generally, a therapeutically effective amount will vary with the subject's age, and condition, as well as the nature and extent of the disease in the subject, all of which can be determined by one of ordinary skill in the art. The dosage may be adjusted by the individual physician, particularly in the event of any complication. A therapeutically effective amount typically varies from 0.01 μg/kg to about 1000 mg/kg, preferably from about 1 μg/kg to about 200 mg/kg and most preferably from about 0.2 mg/kg to about 20 mg/kg, in one or more dose administrations daily, for one or more days. A physician having ordinary skill in the art can readily determine and prescribe the effective amount of the pharmaceutical composition required. For example, the physician could start doses of DAPH and/or the DAPH analog(s) of the invention employed in the pharmaceutical composition at levels lower than that required to achieve the desired therapeutic effect and then gradually increase the dosage until the desired effect is achieved.

The administration of DAPH and/or the DAPH analogs and pharmaceutical compositions as described herein may be administered at various time intervals. In one aspect a single dose may be administered to a subject and provide the desired outcome requiring no further treatment. In other aspects multiple administrations may be required. One of skill in the art is be able to adjust the dosing schedule as required to provide the desired outcome to the subject. In some embodiments, DAPH and/or a DAPH analog(s) or pharmaceutical composition of the invention is provided to a subject chronically. Chronic treatments include any form of repeated administration for an extended period of time, such as repeated administrations for one or more months, between a month and a year, one or more years, or longer. In many embodiments, a chronic treatment involves administering an agent or pharmaceutical composition of the invention repeatedly over the life of the subject. Preferred chronic treatments involve regular administrations, for example one or more times a day, one or more times a week, or one or more times a month. In general, a suitable dose such as a daily dose of an agent of the invention will be that amount of the agent that is the lowest dose effective to produce a therapeutic effect. Such an effective dose will generally depend upon the factors described herein and known to those of skill in the art.

The therapeutics of the invention can be administered by any conventional route, including injection or by gradual infusion over time. The administration may, for example, be oral, intravenous, intracranial, intraperitoneal, intramuscular, intracavity, intrarespiratory, subcutaneous, or transdermal. The route of administration will depend on the composition of a particular therapeutic preparation of the invention. In one preferred embodiment, the route of administration is oral.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Other delivery systems can include time-release, delayed release or sustained release delivery systems. Such systems can avoid repeated administrations of the active compounds of the invention, increasing convenience to the subject and the physician. Many types of release delivery systems are available and known to those of ordinary skill in the art. They include polymer based systems such as polylactic and polyglycolic acid, polyanhydrides and polycaprolactone; nonpolymer systems that are lipids including sterols such as cholesterol, cholesterol esters and fatty acids or neutral fats such as mono-, di and triglycerides; hydrogel release systems; silastic systems; peptide based systems; wax coatings, compressed tablets using conventional binders and excipients, partially fused implants and the like. In addition, a pump-based hardware delivery system can be used, some of which are adapted for implantation.

A long-term sustained release implant also may be used. “Long-term” release, as used herein, means that the implant is constructed and arranged to deliver therapeutic levels of the active ingredient for at least 30 days, and preferably 60 days. Long-term sustained release implants are well known to those of ordinary skill in the art and include some of the release systems described above. Such implants can be particularly useful in treating neurological conditions characterized by aggregates of protein oligomer and/or protein fiber aggregates, for example, by placing the implant near portions of the brain affected by such aggregates, thereby effecting localized, high doses of the compounds of the invention.

It is envisioned that DAPH and/or the DAPH analogs described herein can be delivered to neuronal cells by site-specific means. Cell-type-specific delivery can be provided by conjugating a compound to a targeting molecule, e.g., one which selectively binds to the affected neuronal cells. Methodologies for targeting include conjugates, such as those described in U.S. Pat. No. 5,391,723 to Priest. Another example of a well-known targeting vehicle is liposomes. Liposomes are commercially available from a variety of suppliers. Numerous methods are published for making targeted liposomes. Liposome delivery can be provided by encapsulating compound in liposomes which include a cell-type-specific targeting molecule. Methods for targeted delivery of compounds to particular cell types are well-known to those of skill in the art.

In some circumstances, for example for treatment of neurological protein aggregation disorders, it may be preferred to conjugate DAPH and/or the DAPH analogs to a molecule which facilitates transport of the compound across the blood-brain barrier (BBB). As used herein, a molecule which facilitates transport across the BBB is one which, when conjugated to the compound, facilitates the amount of compound delivered to the brain as compared with non-conjugated compound. The molecule can induce transport across the BBB by any mechanism, including receptor-mediated transport, and diffusion. DAPH and/or the DAPH analogs can be conjugated to such molecules by well-known methods, including bifunctional linkers, and formation of biotin/streptavidin or biotin/avidin complexes by attaching either biotin or streptavidin/avidin to the compound and the complementary molecule to the BBB-transport facilitating molecule.

Molecules which facilitate transport across the BBB include transferrin receptor binding antibodies (U.S. Pat. No. 5,527,527); certain lipoidal forms of dihydropyridine (see, e.g., U.S. Pat. No. 5,525,727); carrier peptides, such as cationized albumin or Metenkephalin (and others disclosed in U.S. Pat. Nos. 5,442,043; 4,902,505; and 4,801,575); cationized antibodies (U.S. Pat. No. 5,004,697); and fatty acids such as docosahexanoic acid (DHA; U.S. Pat. No. 4,933,324).

For other uses of DAPH and/or the DAPH analogs, it may be preferred to administer DAPH and/or the DAPH analogs in combination with a molecule which increases transport of compounds across the blood-brain barrier (BBB). Such molecules, which need not be conjugated to a compound, increase the transport of the compound across the BBB into the brain. A molecule which increases transport across the BBB is one, for example, which increases the permeability of the BBB, preferably transiently. Coadministration of a compound with such a molecule permits the compound to cross a permeabilized BBB. Examples of such molecules include bradykinin and agonist derivatives (U.S. Pat. No. 5,112,596); and receptor-mediated permeabilizers such as A-7 (U.S. Pat. Nos. 5,268,164 and 5,506,206).

### EXAMPLES

### Example 1

### Effect of DAPH and DAPH Analogs on Aggregation

Methods

NM Aggregation Assay

Soluble Sup35 NM protein (“NM”) was tested for aggregation in the presence of DAPH, DAPH analogs or vehicle control (DMSO). NM protein (5 μM) was aggregated in the presence of DAPH (0-20 μM), DAPH analogs (0-20 μM) or DMSO (0-2% v/v). The assay mixture was incubated for 24 hr at 25° C., then tested for Congo Red binding to determine the extent of NM fiber formation.

NM Disaggregation Assay

For determining disaggregation of preformed aggregates, NM protein fibers (equivalent to 5 μM monomer) were incubated with DAPH (0-20 μM), DAPH analogs (0-20 μM) or DMSO (0-2% v/v). The assay mixture was incubated for 24 hr at 25° C., then tested for Congo Red or Thioflavin T binding to determine the extent of NM fibers remaining.

NM Seed Assay

Monomers of NM disaggregated by treatment with DAPH and DAPH analogs (see below) were tested for the ability to “seed” new NM fibers. Disaggregated monomers were used at 2% or 5% as described above for the NM aggregation assay.

Aβ Peptide and Compound Preparation

Stock solutions of Aβ42 (special TFA preparation, catalog no. 03-112, BioSource International, Camarillo, Calif.) were prepared in autoclaved water with the addition of 1 M NaOH to pH 10-11.

Aβ42_{Total} is unfractionated synthetic Aβ42 that was dissolved in water at pH 8-9 by using NH₃ or at pH 10-11 by using dilute NaOH and stored at −40° C.

Aβ42_{30K} is seedless Aβ42 made according to the protocols of Fezoui et al. (Amyloid 7:166-178, 2000), except that the dissolved peptide was filtered through a 30,000-kDa spin filter instead of a 10,000-kDa filter. The method involves dissolving the total Aβ42 peptide in water at pH 10.5 by using NaOH. There was considerable loss of peptide; the concentration of peptide in the filtrate was determined by the intrinsic fluorescence of the single tyrosine residue [Eₓ=280 nm; Eₘ=310 nm, where Eₓ, and Eₘ are the excitation and emission wavelengths, respectively, by using tyrosinamide as the standard.

Experimental samples were prepared by diluting the stock solution of Aβ42 to 10 μM (unless noted otherwise) in Tyrode's solution 2 mM Ca (150 mM NaCl/3 mM KCl/10 mM Hepes, pH 7.4/2 mM CaCl₂/10 mM D-glucose, pH 7.4/0.02% Na azide). The synthetic Aβ42 was dissolved in water at a pH of ˜8 and then stored at −20° C. until diluted into Tyrode's Solution/2 mM Ca (pH 7.4). This preparation is “Aβ42_{Total}”. To aggregate for use in experiments, it was preincubated for 24 or 48 h at 37° C. without stirring.

DAPH (D210; Sigma) and DAPH analogs described herein (DAPH-2-DAPH-13) were prepared in DMSO.

Congo Red Binding

Congo red was added to NM aggregation, NM disaggregation or NM seed reaction mixtures following incubation to a final concentration of 10 μM. After 30 min at 25° C., absorbances at 320, 477, and 540 nm were determined. Congo red dye binding was measured by using the equation [(OD₅₄₀/25,295)−(OD₄₇₇/46,306)] (Klunk et al., J. Histochem. Cytochem. 37:1273-1279, 1989) as described by Schirmer and Lindquist (Proc. Natl. Acad. Sci USA 94:13932-13937, 1997).

Thioflavin T (ThT) Binding

To measure β-sheet formation, ThT was added to protein or peptide reaction mixtures, such as Aβ samples or NM aggregation, NM disaggregation or NM seed reaction mixtures following incubation and fluorescent measurements were read according to standard procedures.

For measurement, each sample preferably is split into multiple wells of a 96-well black-bottom plate (e.g., catalog no. 35-3943, VWR International, Westchester, Pa.). ThT fluorescence is measured at room temperature (e.g., in a Fluoroskan II at Em=444 nm and Ex=510 nm or a FLUOstar Optima plate-reader (BMG Lab Technologies, Durham, N.C.) at Ex=440 nm and Em=480 nm). The ThT fluorescence spectrum is measured in an spectrofluorimeter (e.g., f4500, Hitachi, Tokyo) at Ex=435 nm and Em=450-550 nm.

Electron Microscopy (EM)

Negative-staining EM was used to visualize the kinetics and morphology of Aβ42 fibrillization over time, with and without the presence of DAPH analogs. Samples of 10 μM seedless Aβ42 (Aβ42₃₀ₖ) were incubated for a predetermined amount of time at 37° C. and vigorously vortex mixed immediately before and after incubation. Very small volumes, typically 5 μl, of each sample were absorbed for 2-4 min onto glow-discharged, carbon-coated, Formvar-filmed 400 mesh copper grids and gently wicked away with filter paper. A total of 5 μl of freshly filtered 2% uranyl acetate staining solution was then absorbed for 2 min onto the grid and gently wicked off. Grids were allowed to dry in a light-protected environment overnight before being viewed in a 1200 EXII EM (JEOL) operated at 80 kV. Images were captured on EM film, and positives were printed.

EM Image Analysis

Images were scanned for fiber measurement in NIH IMAGE 1.62. Fiber measurements were calibrated by comparison with the pixel lengths of T4 phage tails, which are known to be 100 nm long. Measurements were analyzed, and width-distribution histograms were produced by using EXCEL 98 (Microsoft).

Kinase Assay

To determine the effect of DAPH or DAPH analogs on kinase activity, standard kinase assays were employed.

Results

Aβ Peptide

The Aβ peptide was allowed to aggregate at pH 7.4 for 24 h and thus to form fibrils. The incubation was either continued for another 24 h in the presence of the vehicle (1% DMSO) or in the presence of equimolar DAPH or DAPH analogs that had been dissolved in DMSO. Treatment with DAPH and certain DAPH analogs reversed the aggregation of the Aβ fibrils as determined by EM.

Fibrils of aggregated Aβ42_{Total} give a strong ThT fluorescence signal, indicating high β-sheet content. Reversal of the fibrils, as detected by the ThT assay, takes only very few minutes and depends on the concentration of DAPH or DAPH analogs (e.g., DAPH-12).

Coincubation of Aβ42₃₀ₖ with equimolar DAPH (10:10 μM) or DAPH analogs resulted in almost complete elimination of the usual fibrils, as observed by EM. The vehicle DMSO (1%) was present in the test and control experiments. EM studies reveal a nearly complete elimination of higher-order structure upon 24 h incubation with DAPH and certain DAPH analogs.

NM Protein

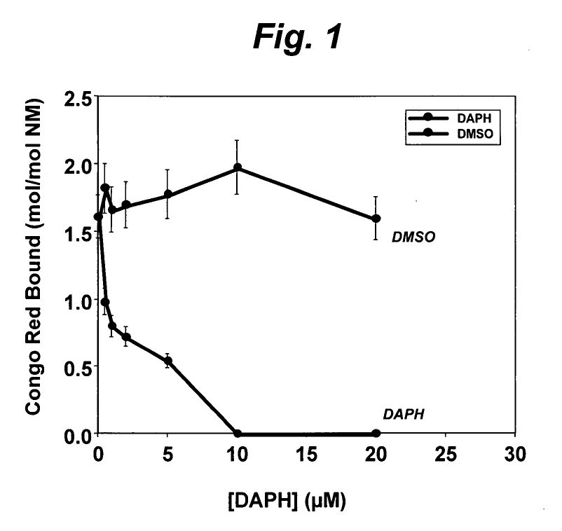

Soluble Sup35 NM (“NM”) at 5 μM was tested for aggregation in the presence of DAPH, DAPH analogs or vehicle control (DMSO). Aggregation of NM was inhibited by up to 100% (see FIG. 1). Inhibition of NM aggregation was concentration dependent.

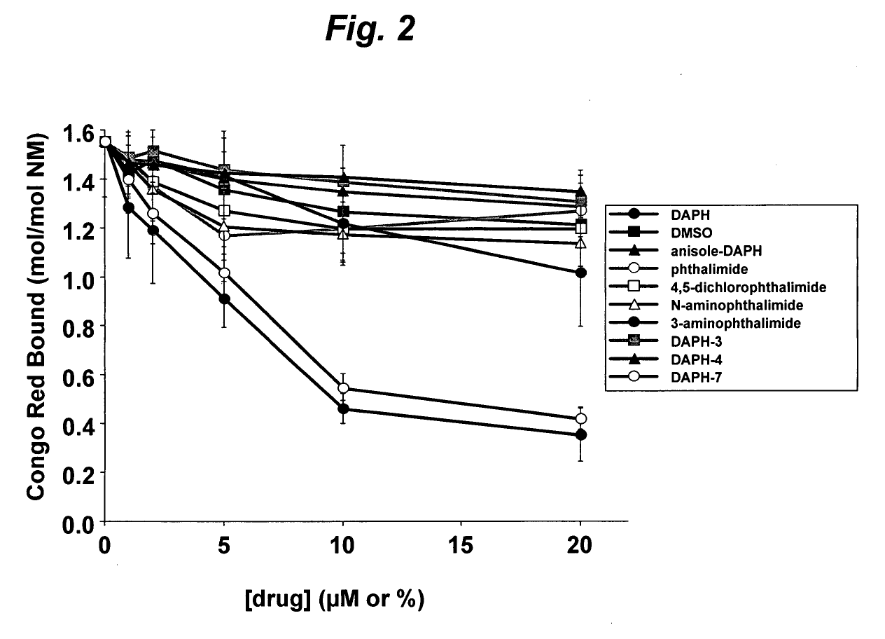

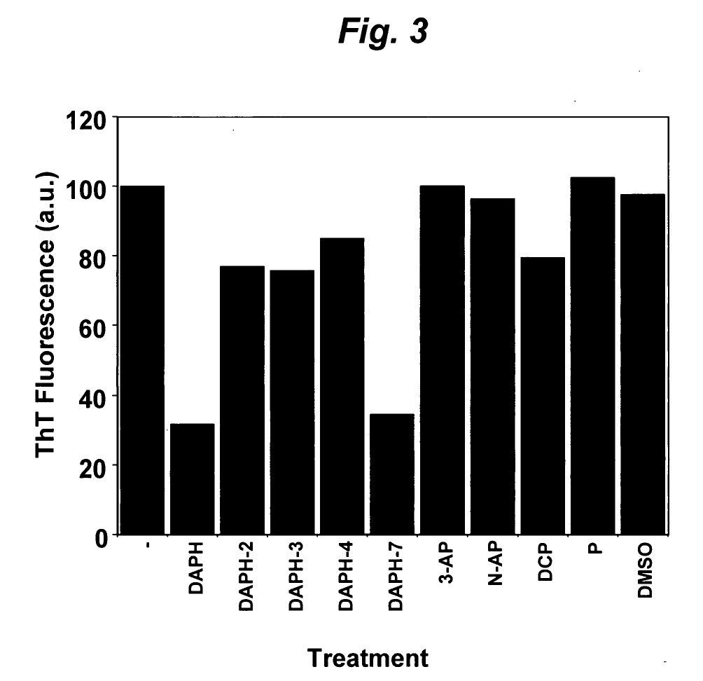

A similar assay was performed using preformed NM aggregates. DAPH and certain DAPH analogs were effective in disaggregation of preformed fibers as compared to the DMSO control (see FIGS. 2 and 3).

In the NM seed assay, NM protein was essentially unable to reform NM fibers following disaggregation with DAPH; certain DAPH analogs exhibited modest effects.

Bisindolylmaleimide IV (DAPH-13) had a similar effect on NM aggregation.

Additional experiments using α-synuclein and polyglutamine (as a model for huntingtin and other polyglutamine proteins) demonstrated a similar effect of DAPH and bisindolylmaleimide IV (DAPH-13).

### Example 2

### Effect of DAPH and DAPH-12 Analog on Prion Disease Model

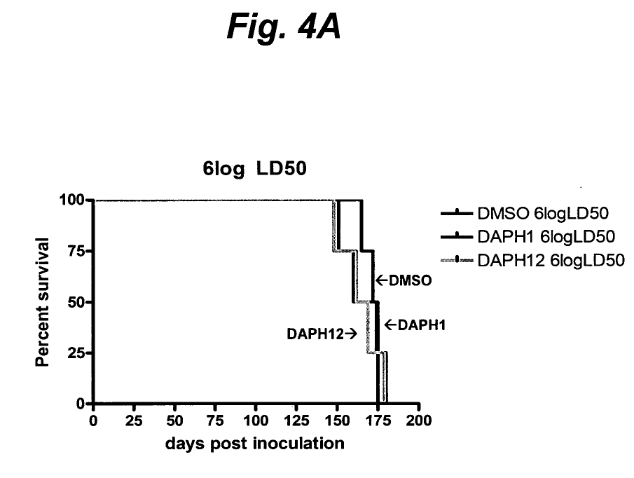

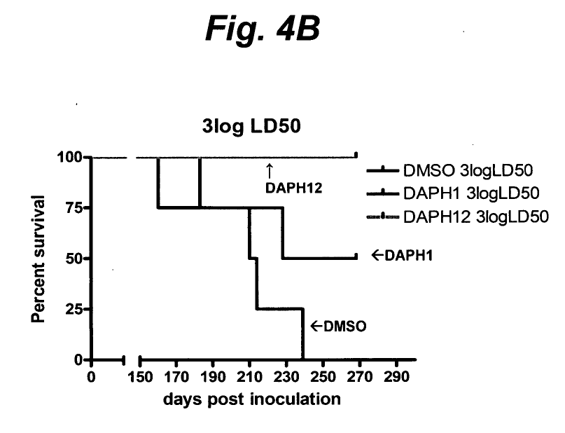

1% brain homogenates from mice infected with the RML strain of mouse scrapie were incubated for 48 hours with DMSO, DAPH (DAPH-1), or DAPH-12. Serial dilutions of the homogenate were injected into CD1 mice (n=4 per group) and the survival of these mice is plotted in Kaplan-Meier survival plots (FIGS. 4A and 4B).

At high doses of infectivity, a significant effect of DAPH treatment was not observed, but at lower doses promising results were obtained. DAPH12 treatment led to a reduction in prion titers in that the median survival of mice injected with DAPH12 treated homogenate have a median survival at least 60 days longer than DMSO controls (P=0.007, log rank test). DAPH1 did not significantly alter survival (P=0.18).

Equivalents

Those skilled in the art will recognize, or be able to ascertain using no more than routine experimentation, many equivalents to the specific embodiments of the invention described herein. Such equivalents are intended to be encompassed by the following claims.

All references disclosed herein are incorporated by reference in their entirety.

We claim:

1. 5,6-Bis-(phenylamino)-2-(4-methoxy-phenyl)-isoindole-1,3-dione (DAPH-2) having the structure: 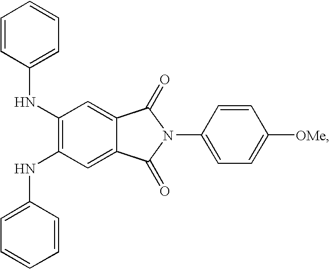
or 5,6-Bis-(2-chloro-phenylamino)-2-(4-methoxyphenyl)-isoindole-1,3-dione (DAPH-3) having the structure: 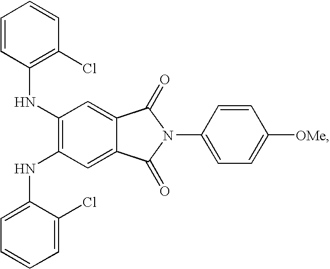
or 5,6-Bis-(3,5-dimethyl-phenylamino)-2-(4-methoxyphenyl)-isoindole-1,3-dione (DAPH-4) having the structure: 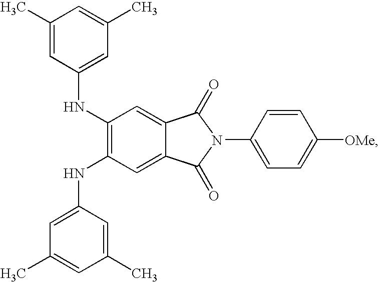
or 5,6-Bis-(4-fluoro-phenylamino)-isoindole-1,3-dione (DAPH-7) having the structure: 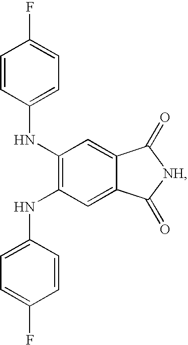
or 5,6-Bis-(4-nitro-phenylamino)-isoindole-1,3-dione (DAPH-10) having the structure: 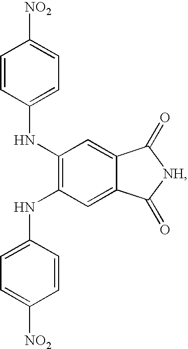
or 4,5-Bis-phenylamino-phthalic acid dimethyl ester (DAPH-11) having the structure: 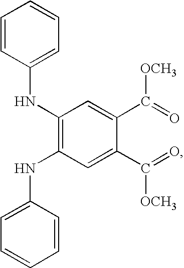
or 5,6-Bis-(4-methoxy-phenylamino)-isoindole-1,3-dione (DAPH-12) having the structure: 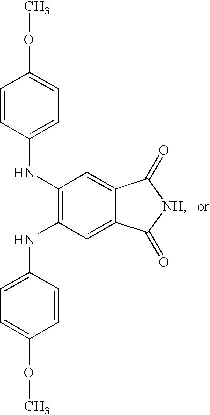
a salt or a solvate thereof.

2.-7. (canceled)

8. A pharmaceutical composition comprising any one or more of the compounds of claim 1, and a pharmaceutically acceptable carrier.

9. A method for disaggregating protein oligomers and/or protein fibers, comprising,
contacting protein fibers with DAPH or one or more DAPH analogs, salts thereof and solvates thereof, in an amount effective to disaggregate the protein oligomers or the protein fibers,
optionally wherein the disaggregation reduces toxicity of the protein oligomers and/or protein fibers,
optionally wherein 4,5-dianilinophthalimide (DAPH), a salt thereof or a solvate thereof is used,
optionally wherein the DAPH analog is 5,6-Bis-(4-fluoro-phenylamino)-isoindole-1,3-dione (DAPH-7), 5,6-Bis-(4-methoxy-phenylamino)-isoindole-1,3-dione (DAPH-12), bisindolylmaleimide IV (DAPH-13), a salt thereof or a solvate thereof,
optionally wherein the protein is selected from the group consisting of amyloid β (Aβ), huntingtin, PrP prion, α-synuclein, cytoplasmic polyadenylation element binding protein (CPEB), yeast Sup35 NM protein (PSI⁺ prion), synphilin, transthyretin, tau, ataxin 1, ataxin 3, atrophin, cystatin B, cystic fibrosis transmembrane conductance regulator (CFTR), the variable domain of immunoglobulin light chains, insulin, leptin, α-lactalbumin, phosphodiesterase γ, prothymosin α and apo-lipoprotein E4, and androgen receptor, and/or
optionally wherein the protein fibers are amyloid fibrils.

10.-16. (canceled)

17. A method for inhibiting aggregation of protein oligomers and/or protein fibers, comprising,
contacting protein oligomers and/or protein fibers with DAPH or one or more DAPH analogs, salts thereof and solvates thereof, in an amount effective to disaggregate the protein oligomers or the protein fibers,
optionally wherein the inhibition of aggregation reduces toxicity of the protein oligomers and/or protein fibers,
optionally wherein 4,5-dianilinophthalimide (DAPH), a salt thereof or a solvate thereof is used,
optionally wherein the DAPH analog is 5,6-Bis-(4-fluoro-phenylamino)-isoindole-1,3-dione (DAPH-7), 5,6-Bis-(4-methoxy-phenylamino)-isoindole-1,3-dione (DAPH-12), or bisindolylmaleimide IV (DAPH-13), or a salt thereof or a solvate thereof,
optionally wherein the protein is selected from the group consisting of amyloid γ (Aγ), huntingtin, PrP prion, α-synuclein, cytoplasmic polyadenylation element binding protein (CPEB), yeast Sup35 NM protein (PSI⁺ prion), synphilin, transthyretin, tau, ataxin 1, ataxin 3, atrophin, cystatin B, cystic fibrosis transmembrane conductance regulator (CFTR), the variable domain of immunoglobulin light chains, insulin, leptin, α-lactalbumin, phosphodiesterase γ, prothymosin α and apo-lipoprotein E4, and androgen receptor, and/or
optionally wherein the protein fibers are amyloid fibrils.

18.-24. (canceled)

25. A method for disaggregating or inhibiting aggregation of protein oligomers and/or protein fibers, comprising
contacting protein oligomers and/or protein fibers with (a) 4,5-dianilinophthalimide (DAPH), salts thereof or solvates thereof and (b) one or more DAPH analogs, salts thereof or solvates thereof, in a combined amount effective to disaggregate and/or inhibit aggregation of the protein oligomers or the protein fibers,
optionally wherein the disaggregation and/or inhibition of aggregation reduces toxicity of the protein oligomers and/or protein fibers, optionally wherein the DAPH analog is 5,6-Bis-(4-fluoro-phenylamino)-isoindole-1,3-dione (DAPH-7), 5,6-Bis-(4-methoxy-phenylamino)-isoindole-1,3-dione (DAPH-12), bisindolylmaleimide IV (DAPH-13), or a salt thereof or a solvate thereof,
optionally wherein the protein is selected from the group consisting of amyloid β (Aβ), huntingtin, PrP prion, α-synuclein, cytoplasmic polyadenylation element binding protein (CPEB), yeast Sup35 NM protein (PSI⁺ prion), synphilin, transthyretin, tau, ataxin 1, ataxin 3, atrophin, cystatin B, cystic fibrosis transmembrane conductance regulator (CFTR), the variable domain of immunoglobulin light chains, insulin, leptin, α-lactalbumin, phosphodiesterase γ, prothymosin α and apo-lipoprotein E4, and androgen receptor, and/or
optionally wherein the protein fibers are amyloid fibrils.

26.-38. (canceled)

39. A method for treating a subject having a protein aggregation disorder, comprising
administering to a subject in need of such treatment DAPH or one or more DAPH analogs, salts thereof and solvates thereof, in an amount effective to disaggregate protein oligomers or protein fibers and/or to inhibit aggregation of protein oligomers and/or protein fibers,
optionally wherein the disaggregation and/or inhibition of aggregation reduces toxicity of the protein oligomers and/or protein fibers,
optionally wherein 4,5-dianilinophthalimide (DAPH), a salt thereof or a solvate thereof is administered,
optionally wherein the DAPH analog is 5,6-Bis-(4-fluoro-phenylamino)-isoindole-1,3-dione (DAPH-7), 5,6-Bis-(4-methoxy-phenylamino)-isoindole-1,3-dione (DAPH-12), or bisindolylmaleimide IV (DAPH-13), or a salt thereof or a solvate thereof,
optionally wherein the protein is selected from the group consisting of amyloid β (Aβ) huntingtin, PrP prion, α-synuclein, cytoplasmic polyadenylation element binding protein (CPEB), yeast Sup35 NM protein (PSI⁺ prion), synphilin, transthyretin, tau, ataxin 1, ataxin 3, atrophin, cystatin B, cystic fibrosis transmembrane conductance regulator (CFTR), the variable domain of immunoglobulin light chains, insulin, leptin, α-lactalbumin, phosphodiesterase γ, prothymosin α and apo-lipoprotein E4, and androgen receptor,
optionally wherein the protein fibers are amyloid fibrils,
optionally wherein the protein aggregation disorder is selected from the group consisting of Alzheimer's disease; tauopathy; α-synucleinopathy; Parkinson's disease; Huntington's Disease; prion related diseases; Type II Diabetes Mellitus associated with islet amyloid polypeptide (IAPP); and neurodegenerative diseases associated with intracellular and/or intraneuronal aggregates of proteins with polyglutamine, polyalanine or other repeats arising from pathological expansions of tri- or tetra-nucleotide elements within corresponding genes, and/or
optionally wherein the subject is free of symptoms otherwise calling for treatment with the composition.

40.-58. (canceled)

59. A method for treating a subject having a protein aggregation disorder, comprising
administering to a subject in need of such treatment (a) 4,5-dianilinophthalimide (DAPH), salts thereof or solvates thereof and (b) one or more DAPH analogs, salts thereof or solvates thereof, in a combined amount effective to disaggregate or to inhibit aggregation of protein oligomers and/or protein fibers,
optionally wherein the disaggregation and/or the inhibition of aggregation reduces toxicity of the protein oligomers and/or protein fibers,
optionally wherein the DAPH analog is 5,6-Bis-(4-fluoro-phenylamino)-isoindole-1,3-dione (DAPH-7), 5,6-Bis-(4-methoxy-phenylamino)-isoindole-1,3-dione (DAPH-12), or bisindolylmaleimide IV (DAPH-13), or a salt thereof or a solvate thereof,
optionally wherein the protein is selected from the group consisting of amyloid β (Aβ), huntingtin, PrP prion, α-synuclein, cytoplasmic polyadenylation element binding protein (CPEB), yeast Sup35 NM protein (PSI⁺ prion), synphilin, transthyretin, tau, ataxin 1, ataxin 3, atrophin, cystatin B, cystic fibrosis transmembrane conductance regulator (CFTR), the variable domain of immunoglobulin light chains, insulin, leptin, α-lactalbumin, phosphodiesterase γ, prothymosin α and apo-lipoprotein E4, and androgen receptor,
optionally wherein the protein fibers are amyloid fibrils,
optionally wherein the protein aggregation disorder is selected from the group consisting of Alzheimer's disease; tauopathy: α-synucleinopathy; Parkinson's disease: Huntington's Disease: prion related diseases: Type II Diabetes Mellitus associated with islet amyloid polypeptide (IAPP): and neurodegenerative diseases associated with intracellular and/or intraneuronal aggregates of proteins with polyglutamine, polyalanine or other repeats arising from pathological expansions of tri- or tetra-nucleotide elements within corresponding genes, and/or
optionally wherein the subject is free of symptoms otherwise calling for treatment with the composition.

60.-76. (canceled)

77. A composition comprising
4,5-dianilinophthalimide (DAPH), salts thereof or solvates thereof, and
one or more DAPH analogs, salts thereof or solvates thereof,
optionally comprising a pharmaceutically acceptable carrier,
optionally wherein the compounds are present in a combined amount effective to inhibit aggregation of protein oligomers and/or protein fibers or disaggregate protein oligomers and/or protein fibers,
optionally wherein the DAPH analog is 5,6-Bis-(4-fluoro-phenylamino)-isoindole-1,3-dione (DAPH-7), 5,6-Bis-(4-methoxy-phenylamino)-isoindole-1,3-dione (DAPH-12), or bisindolylmaleimide IV (DAPH-13), or a salt thereof or a solvate thereof,

78.-82. (canceled)

83. A method for inhibiting biofilm formation comprising
contacting an organism that forms a biofilm with DAPH and/or one or more DAPH analogs, salts thereof and solvates thereof, in an amount effective to inhibit aggregation of protein oligomers or protein fibers in the biofilm,
optionally wherein 4,5-dianilinophthalimide (DAPH), a salt thereof or a solvate thereof is used,
optionally wherein the DAPH analog is 5,6-Bis-(4-fluoro-phenylamino)-isoindole-1,3-dione (DAPH-7), 5,6-Bis-(4-methoxy-phenylamino)-isoindole-1,3-dione (DAPH-12), or bisindolylmaleimide IV (DAPH-13), or a salt thereof or a solvate thereof.

84.-87. (canceled)

88. A method for disaggregating biofilms comprising
contacting the biofilm with DAPH and/or one or more DAPH analogs, salts thereof and solvates thereof, in an amount effective to disaggregate the biofilm,
optionally wherein 4,5-dianilinophthalimide (DAPH), a salt thereof or a solvate thereof is used,
optionally wherein the DAPH analog is 5,6-Bis-(4-fluoro-phenylamino)-isoindole-1,3-dione (DAPH-7), 5,6-Bis-(4-methoxy-phenylamino)-isoindole-1,3-dione (DAPH-12), or bisindolylmaleimide IV (DAPH-13), or a salt thereof or a solvate thereof.

89.-92. (canceled)
